SEQUENCE LISTING (5 sequences)

SEQ ID NO: 1 - protein, 10 aa, synthetic construct
TQTNSPRRAR

SEQ ID NO: 2 - protein, 15 aa, synthetic construct
CASYQTQTNSPRRAR

SEQ ID NO: 3 - protein, 17 aa, synthetic construct
CASYQTQTNSPRRARSV

SEQ ID NO: 4 - protein, 18 aa, synthetic construct
ASYQTQTNSPRRARSVAS

SEQ ID NO: 5 - protein, 159 aa, synthetic construct
MSYTIATPSQFVFLSSAWADPIELINLCTNSLGNQFQTQQARTTVQRQFSEVWKPVPQVTVRFPDSGFKVYRYNAVLDPLVTALLGAFDTRNRIIEVENQANPTTAETLDATRRVDDATVAIRSAINNLVVELVKGTGLYNQSTFESASGLQWSSAPAS

(19) United States
(12) Patent Application Publication
(10) Pub. No.: US 2023/0190918 A1
(43) Pub. Date: Jun. 22, 2023

(54) PICKERING EMULSION-BASED VACCINES

(71) Applicants: The State of Israel, Ministry of Agriculture & Rural Development, Agricultural Research, Rishon Lezion (IL); Yissum Research Development Company of the Hebrew University of Jerusalem Ltd., Jerusalem (IL)

(72) Inventors: Guy MECHREZ, Adi (IL); Meital RECHES, Jerusalem (IL); Aviv DOMBROVSKY, Rishon Lezion (IL)

(21) Appl. No.: 17/961,147

(22) Filed: Oct. 6, 2022

Related U.S. Application Data
(63) Continuation of application No. PCT/IL2021/050390, filed on Apr. 6, 2021.
(60) Provisional application No. 63/005,847, filed on Apr. 6, 2020.

(51) Int. Cl. A61K 39/215; A61P 31/14

(52) U.S. Cl. CPC A61K 39/215; A61P 31/14; A61K 2039/55566

(57) ABSTRACT

A particle comprising a shell comprising an immunogenic nanoparticle bound to at least one epitope and in contact with the shell, is provided. An emulsion comprising a plurality of said particles is also provided, such as for vaccinating a subject in need thereof.

### CROSS REFERENCE TO RELATED APPLICATIONS

This application is a Bypass Continuation of PCT Patent Application No. PCT/IL2021/050390 having International filing date of Apr. 6, 2021, which claims the benefit of priority of U.S. Provisional Patent Application No. 63/005,847 filed on Apr. 6, 2020, the contents of which are all incorporated herein by reference in its entirety.

### FIELD OF THE INVENTION

The present invention relates in general to the field of vaccines.

### BACKGROUND OF THE INVENTION

The recent emergence of SARS-CoV-2 pandemic has emphasized the necessity of rapid vaccine development strategies, which could be readily accommodated to pathogen variations. The current vaccine technologies could be classified into two main categories: (i) Nucleic acid-based vaccines, which include messenger RNAs and DNA plasmids that encode viral antigenic proteins produced by the host cells, as well as viral vector-based vaccines and attenuated live-viruses, and (ii) protein-based vaccines, which are based on presentation of antigenic viral peptides, as well as inactivated whole virus and subunit vaccines.

Subunit vaccines are based on the presentation of one or more viral antigens (e.g., proteins, peptides and carbohydrate antigens) on a carrier that instigate the immune response, without the introduction of a whole pathogen and without any host cell modifications, manifesting the potentially safest vaccine technology that could be applied against viral infections, such as SARS-CoV-2. One of the major challenges in the development of subunit vaccines is the ability to immobilize and expose high amounts of epitopes on the vaccine vector for stimulating a suitable immune response that is required for efficient vaccination.

Pickering emulsions are commonly formed by the self-assembly of colloidal particles at the interface between two immiscible liquids. The origin of the strong anchoring of the nanoparticles at the oil/water (o/w) interface is the partial wetting of the particles' surface by both liquids. Importantly, it has been shown that Pickering emulsions are highly stable and could serve as adjuvants, enhancing the recruitment and activation of antigen-presenting cells.

Developing innovative approaches that could ensure a highly efficient immune response towards the antigenic subunits is challenging and important for immediate applications.

### SUMMARY OF THE INVENTION

The invention provides, in some embodiments, particles and emulsions for use in vaccinating a subject in need thereof. The invention further provides methods of immunizing a subject, and methods for preparing the particles and emulsions described herein.

The present invention is based, in part, on results showing a novel technology for the generation of fully synthetic subunit vaccines. The vaccines include high intensity of the epitope presentation levels, achieved by a two-mode enhancement mechanism, achieving heterogeneity and density of epitope presentation. The first epitope concentration enhancement level is obtained by covalent immobilization of peptide epitopes on the surface of immunogenic innocuous virus-like particles (VLPs) derived from the coat proteins (CPs) of plant viruses, including but not limited to tomato brown rugose fruit virus (ToBRFV) and pepino mosaic virus (PepMV). The second level of epitope concentration enhancement was obtained by assembly of the VLP/epitope conjugates on the surface of paraffin oil droplets at the interface of paraffin-in-water emulsion as Pickering stabilizers (FIG. 1).

According to one aspect, there is provided a particle in a form of a colloidosome comprising a shell and a core comprising an oil, wherein said shell comprises an immunogenic nanoparticle in contact with the core, the nanoparticle being covalently bound to at least one epitope.

According to some embodiments, the particle has a diameter of 10 μm to 100 μm.

According to some embodiments, the shell has a diameter of 10 nm to 100 nm. According to some embodiments, the nanoparticle has a diameter of 10 nm to 100 nm.

According to some embodiments, the particle comprises 1% to 20% (w/w) of said nanoparticles.

According to some embodiments, the immunogenic nanoparticle is virus-like particle. According to some embodiments, the virus-like particle is a plant viruses-like particle. According to some embodiments, the virus-like particle is derived from a coat protein of a virus selected from the group consisting of Tobamovirus, and Potexvirus, or a combination thereof. According to some embodiments, the particle comprises at least two types of immunogenic nanoparticles.

According to some embodiments, the at least one epitope is derived from SARS-CoV-2 spike glycoprotein. According to some embodiments, the at least one epitope comprises the amino acid sequence as set forth in TQTNSPRRAR (SEQ ID NO: 1). According to some embodiments, the at least one epitope is selected from the group consisting of CASYQTQTNSPRRAR (SEQ ID NO: 2); CASYQTQTNSPRRARSV (SEQ ID NO: 3), and ASYQTQTNSPRRARSVAS (SEQ ID NO: 4). According to some embodiments, the at least one epitope comprises N-terminal acetylation.

According to another aspect, there is provided a composition comprising a plurality of particles of the present invention, and a pharmaceutically acceptable carrier. According to some embodiments, the composition is a pharmaceutical composition. According to some embodiments, the composition is an immunogenic composition. According to some embodiments, the composition is an oil-in-water emulsion. According to some embodiments, the composition is for use in treating or preventing an infection (e.g., a viral infection) in a subject in need thereof.

According to another aspect, there is provided a method for treating or preventing an infection (e.g., a viral infection) in a subject in need thereof, comprising administering to said subject a therapeutically effective amount of the composition of the present invention, thereby treating or preventing a viral infection in the subject.

According to another aspect, there is provided a composition of the present invention for use in treating or preventing an infection in a subject in need thereof.

According to another aspect, there is provided a kit comprising the composition of the present invention, such as for use in treating or preventing an infection in a subject in need thereof.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 presents a non-limiting schematic illustration: two-mode enhancement mechanism of epitope presentation. (1) The preparation of immunogenic VLPs based on ToBRFV and PepMV CPs; (2) Synthesis of SARS-CoV-2 epitopes and their covalent immobilization on the VLPs' surface exhibiting the first level of epitope concentration enhancement; (3) The VLP/epitope conjugates are then assembled at the oil/water interface of a Pickering emulsion, obtaining a second-level of epitope concentration enchantment; (4) In-vivo trials of the VLP/epitope-Pickering emulsions for immunogenicity in mice.

FIGS. 2A-2D show purification assessment of ToBRFV and PepMV viral particles in the viral preparation from co-infected tomato plants. Transmission electron microscopy for visualization of virion particle morphologies (2A, B); Western blot analyses detecting ToBRFV and PepMV coat proteins (CPs) in the viral preparation from the co-infected tomato plants (2C); FIG. 2D: I. II., Sequential western blot analyses for cross detection of ToBRFV and PepMV CPs on each of the analysed viral preparations; M, molecular weight ladder; V, viral preparation.

FIGS. 3A-3E depict characteristic confocal fluorescence microscopy images of 20:80 o/w Pickering emulsions stabilized by 1.3 wt % VLPs. ToBRFV CP detection in Pickering emulsions subjected to specific fluorescent antibodies (Alexa Fluor 488) against ToBRFV using the green channel (3A); PepMV CP detection in Pickering emulsions subjected to specific fluorescent antibodies (Alexa Fluor 594) against PepMV using the red channel (3B); Small and high magnifications of the fluorescence signals in Pickering emulsions using combined green and red channels; On the right of the fluorescent images a schematic illustration (3E) of the oil droplets in the VLP stabilized Pickering emulsions is depicted; Scale bars represent 10 μm (3C, D).

FIGS. 4A-4E present characteristic cryogenic HRSEM micrographs of 20:80 o/w Pickering emulsions stabilized by 1.3 wt % VLPs. Pickering emulsions vitrified, fractured and subsequently subjected to a controlled sublimation for interface exposure were analysed. A characteristic basic structure of a Pickering emulsion (4A, B); A higher magnification micrographs (×10) showing the presence of the VLPs at the o/w interface of the oil droplets (4C,D); On the right of the micrographs a schematic illustration (4E) of the oil droplets in the VLP stabilized Pickering emulsions is depicted.

FIGS. 5A-5D present characteristic confocal fluorescence microscopy images of 20:80 o/w Pickering emulsions stabilized by 1.3 wt % VLP/fluorescent epitope conjugates. (5A) A [5(6)-FAM] labelled SARS-CoV-2 S1 epitope visualized on oil droplets with the green channel; (5B) PepMV-CP detection using Alexa Fluor 594 specific fluorescent antibodies, visualized with the red channel; (5C) Co-localization of the green and red fluorescent signals (Shown in orange) visualized with both green and red channels; On the right of the fluorescent images a schematic illustration (5D) of the oil droplets in the Pickering emulsions stabilized by VLP/epitope conjugates is depicted; The scale bar represents 5 μm.

FIGS. 6A-6D present immunization efficiencies and specificity of antisera developed in mice vaccinated by VLP/epitope-based Pickering emulsions designed against SARS-CoV-2-S1 peptide. (6A-C) ELISA tests of antisera produced in mice in response to various vaccine preparations against SARS-CoV-2-S1 peptide; (6D) Dot blot analyses of mouse antisera developed against the synthetic SARS-CoV-2-S1 peptide presented by the VLP/epitope-based Pickering emulations and controls. The first raw: depicts the blotted membranes of mouse sera which were exposed to the VLP/epitope Pickering emulsions prepared by using o/w ratios of 20:80, 30:70, 40:60, and 50:50. The second raw: d1, Naive mouse sera; d2, VLPs (comprised of ToBRFV and PepMV) dissolved in water; d3, peptide epitopes dissolved in water; d4, Peptide epitopes administered with adjuvants; d5, Alkaline phosphatase reagent control; d6, A secondary antibody control.

### DETAILED DESCRIPTION OF THE INVENTION

According to some embodiments, the present invention provides a particle having high concentrations of epitopes bound thereto, for use as a vaccine having increased immunogenicity.

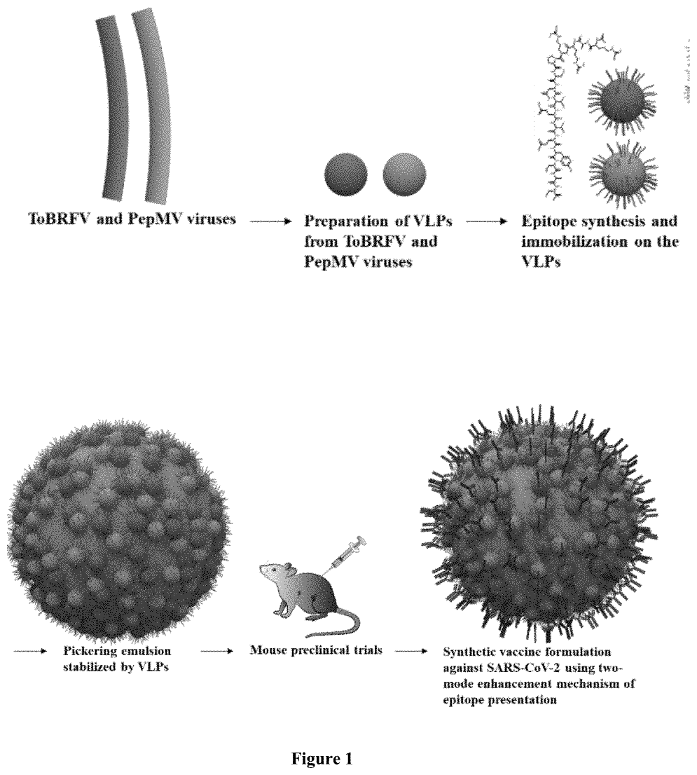

Reference is made to FIG. 1, which presents a schematic illustration of an immunogenic colloidosome forming Pickering emulsion, according to some embodiments of the present invention. The present invention is based, in part, on a novel approach in vaccine development implemented for the rapid development of a vaccine (including but not limited to COVID-19 virus) based on antigenic determinants (epitopes) that are introduced to the blood circulation at very high concentrations and increased immunogenicity.

As demonstrated herein, the inventors present a novel technology for the generation of fully synthetic subunit vaccines. Successful immunization against SARS-CoV-2 S1 in mice was achieved. The high intensity of the epitope presentation levels was achieved by a two-mode enhancement mechanism, achieving heterogeneity and density of epitope presentation. The first mode engaged a high epitope concentration of SARS-CoV-2 S1 peptide epitope covalently immobilized on the VLPs surface. The second mode is achieved by the assembly of the VLPs/epitope conjugates on the surface of oil droplets at an oil/water interface of an emulsion as Pickering stabilizers. This two-mode epitope display system provoked a higher IgG titer in mice compared to the classical adjuvant-associated immunization. ToBRFV and PepMV based VLPs are characterized with very high stability which opens up the possibility to develop safe vaccine technologies with improved efficiency and shelf life against various pathogens, including but not limited to, SARS-CoV-2. The described platform is highly flexible and by using multiple epitopes can be easily applied and extended for immunization against a wide range of pathogen-epitopes.

According to some embodiments, there is provided an emulsion comprising a plurality of particles. In some embodiments, the composition is an oil-in-water (O/W) Pickering emulsion. According to some embodiments, the emulsion is for use in vaccinating a subject in need thereof. The present invention further concerns methods of treating and preventing infections and methods of generating antibodies.

According to some embodiments, there is provided a particle comprising a shell and a core, wherein (i) the core comprises an oil, and (ii) the shell being a plurality of immunogenic nanoparticles in contact with the core, each nanoparticle being bound to a plurality of epitopes.

According to some embodiments, the invention provides an immunogenicity-tunable particle. In some embodiments, by virtue of selecting the type and amounts (e.g. loading amounts) of immunogenic particles (e.g., VLP), and type and amounts of epitopes bound to each immunogenic particle, a tunable immunogenic particle is achieved. In some embodiments, the selection of the oil forming the core (e.g., a specific mineral oil) can further serve as to enhance the immunogenicity of the particle.

In some embodiments, the nanoparticle being covalently bound to at least one epitope. In some embodiments, the nanoparticle being non-covalently (e.g., electrostatic interaction, hydrophobic interaction etc.) bound to at least one epitope.

According to some embodiments, the immunogenic nanoparticle is derived from a plant virus. According to some embodiments, the immunogenic nanoparticle is derived from a coat protein (CP) of a virus selected from the group consisting of Tobamovirus, and Potexvirus, or a combination thereof. According to some embodiments, the CP of Tobamovirus is ToBRFV CP.

According to some embodiments, the immunogenic nanoparticle is virus-like particle (VLP). According to some embodiments, the virus-like particle is a plant viruses-like particle. According to some embodiments, the virus-like particle is derived from a coat protein (CP) of a virus selected from the group consisting of Tobamovirus, and Potexvirus, or a combination thereof. According to some embodiments, the CP of Tobamovirus is ToBRFV CP

According to some embodiments, the ToBRFV CP has a GenBank accession number of KX619418.

According to some embodiments, the ToBRFV CP has an amino acid sequence as set forth in:

(SEQ ID NO: 5)
MSYTIATPSQFVFLSSAWADPIELINLCTNSLGNQFQTQ
QARTTVQRQFSEVWKPVPQVTVRFPDSGFKVYRYNAVLD
PLVTALLGAFDTRNRIIEVENQANPTTAETLDATRRVDD
ATVAIRSAINNLVVELVKGTGLYNQSTFESASGLQWSSA
PAS.

According to some embodiments, the particle comprises at least two types of immunogenic nanoparticles.

According to some embodiments, the at least two types of immunogenic nanoparticles is at least two types of coat protein (CP), such as derived from two different sources of virus-like particles. According to some embodiments, the at least two types of immunogenic nanoparticles is at least CP from Tobamovirus, and at least one CP from Potexvirus.

According to some embodiments, the at least two types of immunogenic nanoparticles is at least one VLP covalently bound to a first epitope, and at least one additional VLP covalently bound to a second epitope.

According to some embodiments, the immunogenic nanoparticle is a synthetic particle. A “synthetic particles” as used herein, is a particle that is formed by a chemical or physical process, preferably monomer polymerization, polymer precipitation, macromolecular bond assembly, e.g., aggregation or thermal denaturation, and the re-assembled.

According to some embodiments, the at least one epitope is derived from a spike glycoprotein. According to some embodiments, the at least one epitope is derived from SARS spike glycoprotein. According to some embodiments, the at least one epitope is derived from SARS-CoV-2 spike glycoprotein.

According to some embodiments, the at least one epitope comprises the amino acid sequence as set forth in TQTNSPRRAR (SEQ ID NO: 1). According to some embodiments, the at least one epitope is selected from the group consisting of CASYQTQTNSPRRAR (SEQ ID NO: 2); CASYQTQTNSPRRARSV (SEQ ID NO: 3), and ASYQTQTNSPRRARSVAS (SEQ ID NO: 4).

According to some embodiments, the epitope is a peptide comprising at least one post-translational modification. In some embodiments, the peptide comprises at least one post-translational modification at the N- or C-termini of said peptide. In some embodiments, the peptide comprises at least one post-translational modification (including but not limited to acetylation and amidation). In some embodiments, the peptide is acetylated. In some embodiments, the N-terminus of the peptide is capped or protected (e.g., acetylated) such as not to allow reaction between carboxy group the various peptides on the nanoparticle. In some embodiments, the N-terminus of the peptide is acetylated. According to some embodiments, the at least one epitope comprises N-terminal acetylation.

### Compositions

According to some embodiments, there is provided a composition comprising a plurality of particles of the invention and a pharmaceutically acceptable carrier.

According to some embodiments, the composition is an emulsion or dispersion. According to some embodiments, the composition is an oil-in-water (O/W) Pickering emulsion. According to some embodiments, the composition is an oil-in-oil Pickering emulsion. According to some embodiments, the composition is a water-in-oil (W/O) Pickering emulsion.

In some embodiments, the nanoparticles are in the interface of a major phase and a minor phase, wherein the emulsion is stabilized by the nanoparticles.

As used herein, the term “Pickering emulsion” refers to an emulsion that utilizes solid particles as a stabilizer to stabilize droplets of a substance, in a dispersed phase in the form of droplets dispersed throughout a continuous phase.

As used herein, the term “emulsion” refers to a combination of at least two fluids, where one of the fluids is present in the form of droplets in the other fluid. The term “emulsion” includes microemulsions.

As used herein, the term “fluid” refers to a substance that tends to flow and to conform to the outline of its container, i.e., a liquid, a gas, a viscoelastic fluid, etc. Typically, fluids are materials that are unable to withstand a static shear stress, and when a shear stress is applied, the fluid experiences a continuing and permanent distortion. The fluid may have any suitable viscosity that permits flow. If two or more fluids are present, each fluid may be independently selected among essentially any fluids (liquids, gases, and the like) by those of ordinary skill in the art, by considering the relationship between the fluids. In some cases, the droplets may be contained within a carrier fluid, e.g., a liquid.

In some embodiments, the composition comprises a solvent, selected from an aqueous solvent, a lipophilic organic solvent and a polar organic solvent or any combination thereof.

In some embodiments, the composition (e.g. an emulsion) comprises 0.01% to 10% (w/w), 0.01% to 20% (w/w), 0.05% to 10% (w/w), 0.09% to 10% (w/w), 0.1% to 10% (w/w), 0.5% to 10% (w/w), 0.9% to 10% (w/w), 1% to 10% (w/w), 5% to 10% (w/w), 0.01% to 9% (w/w), 0.05% to 9% (w/w), 0.09% to 9% (w/w), 0.1% to 9% (w/w), 0.5% to 9% (w/w), 0.9% to 9% (w/w), 1% to 9% (w/w), 5% to 9% (w/w), 0.01% to 5% (w/w), 0.05% to 5% (w/w), 0.09% to 5% (w/w), 0.1% to 5% (w/w), 0.5% to 5% (w/w), 0.9% to 5% (w/w), or 1% to 5% (w/w), of the particles, including any range therebetween.

In some embodiments, the particle is a core-shell particle. In some embodiments, the shell comprises an inner portion facing the core and an outer portion facing an ambient. In some embodiments, the inner portion is in contact with the core. In some embodiments, the inner portion is bound to the core. In some embodiments, the shell stabilizes the core. In some embodiments, the shell encapsulates the core.

In some embodiments, the particle is in a form of a colloidosome. The term “colloidosome” refers to a structure that has (i) a shell (e.g., a porous shell) defined by a plurality of nano-materials (e.g., the immunogenic nanoparticles) and optionally interstices formed between the nano-materials; and (ii) a core that is defined by the nano-material structured porous shell. In some embodiments, the term “colloidosome” refers to a structure composed of colloidal particles or materials; i.e., at least a portion of the plurality of nano-materials that form the colloidosome are colloidal particles or materials (e.g., can form a stable dispersion in a given liquid medium).

In some embodiments, the particle is substantially solid. In some embodiments, the particle is in a solid form. In some embodiments, the particle is in a form of a droplet.

In some embodiments, the nanoparticle is covalently bound to a carboxylic group (e.g., the C-terminus) of the at least one epitope. In some embodiments, the nanoparticle is covalently bound by an amino group of said nanoparticle to a carboxylic group (e.g., the C-terminus) of the at least one epitope.

In some embodiments, the particle has a spherical geometry or shape. In some embodiments, a plurality of particles is devoid of any characteristic geometry or shape.

In some embodiments, the particle has a diameter between 0.5 μm and 500 μm, between 0.5 μm and 250 μm, 1 μm to 100 μm, 5 μm to 100 μm, 10 μm to 100 μm, 50 μm to 100 μm, 1 μm to 80 μm, 10 μm to 80 μm, 50 μm to 80 μm, 10 μm to 50 μm, 80 μm to 100 μm, 100 μm to 200 μm, 200 μm to 300 μm, 300 μm to 400 μm, 400 μm to 500 μm, 1 μm to 10 μm, 5 μm to 10 μm, 1 μm to 50 μm, 10 μm to 50 μm, 5 μm to 50 μm, or 1 μm to 5 μm, including any range or value therebetween.

In some embodiments, the diameter of the particle described herein, represents an average diameter. In some embodiments, the size of the particle described herein represents an average or median size of a plurality of particles. In some embodiments, the average or the median size of at least e.g., 10%, 20%, 30%, 40%, 50%, 60%, 70%, 80%, 90%, or 95% of the particles, ranges from: 5 μm to 50 μm, 1 μm to 50 μm, 5 μm to 10 μm, including any range therebetween. In some embodiments, the diameter of the particle described herein, is a dry diameter (i.e. a diameter of isolated dried particles). In some embodiments, a plurality of the particles has a uniform size. By “uniform” or “homogenous” it is meant to refer to size distribution that varies within a range of less than e.g., ±60%, ±50%, ±40%, ±30%, ±20%, or ±10%, including any value therebetween.

In some embodiments, the droplets have a diameter of 1 μm to 100 μm, 5 μm to 100 μm, 10 μm to 100 μm, 50 μm to 100 μm, 1 μm to 80 μm, 10 μm to 80 μm, 50 μm to 80 μm, 1 μm to 10 μm, 5 μm to 10 μm, 1 μm to 50 μm, 10 μm to 50 μm, 5 μm to 50 μm, or 1 μm to 5 μm, including any range therebetween.

As used herein, the term “droplet” refers to an isolated portion of a first fluid that is surrounded by a second fluid. It is to be noted that a droplet is not necessarily spherical; but may assume other shapes as well, for example, depending on the external environment. In some embodiments, the droplet has a minimum cross-sectional dimension that is substantially equal to the largest dimension of the channel perpendicular to fluid flow in which the droplet is located. The fluidic droplets may have any shape and/or size. Typically, monodisperse droplets are of substantially the same size. The shape and/or size of the fluidic droplets can be determined, for example, by measuring the average diameter or other characteristic dimension of the droplets. The “average diameter” of a plurality or series of droplets is the arithmetic average of the average diameters of each of the droplets. Those of ordinary skill in the art will be able to determine the average diameter (or other characteristic dimension) of a plurality or series of droplets, for example, using laser light scattering, microscopic examination, or other known techniques. The average diameter of a single droplet, in a non-spherical droplet, is the diameter of a perfect sphere having the same volume as the non-spherical droplet. In some embodiments, the average diameter of a droplet (and/or of a plurality or series of droplets) is, 5 μm to 100 μm, 5 μm to 50 μm, 1 μm to 50 μm, including any range therebetween. In some embodiments, the average diameter of a droplet is a wet diameter (i.e. a particle diameter within a solution).

According to some embodiments, the particle comprises 1% to 20% (w/w) of said nanoparticles. According to some embodiments, the particle comprises 1% to 10% (w/w) of said nanoparticles.

According to some embodiments, the immunogenic nanoparticle is a hydrophobic nanoparticle.

In some embodiments, the shell comprises between 10% and 99%, between 10% and 20%, between 20% and 30%, between 30% and 50%, between 50% and 60%, between 60% and 70%, between 70% and 80%, between 80% and 90%, between 90% and 99%, (w/w) of the nanoparticles.

In some embodiments, the particle comprises between 1% and 90%, between 10% and 99%, between 10% and 20%, between 20% and 30%, between 30% and 50%, between 50% and 60%, between 60% and 70%, between 70% and 80%, between 80% and 90%, between 90% and 99% (w/w) of the nanoparticles. In some embodiments, the core comprises between 1% and 90%, between 1% and 10%, between 1% and 5%, between 5% and 10%, between 10% and 20%, between 20% and 30%, between 30% and 40%, between 40% and 50%, between 50% and 70%, between 70% and 90% (w/w) of a polymer, including any range therebetween. In some embodiments, the core is devoid of a polymer.

In some embodiments, the particle shell comprises a plurality of nanoparticles. In some embodiments, the nanoparticles are hydrophobic. In some embodiments, the outer surface of the nanoparticles is hydrophobic.

In some embodiments, the nanoparticles are characterized by a median particle size of 1 nm to 900 nm. In some embodiments, the nanoparticles is characterized by a median particle size of 2 nm to 600 nm, 2 nm to 550 nm, 2 nm to 520 nm, 2 nm to 500 nm, 2 nm to 480 nm, 2 nm to 450 nm, 2 nm to 400 nm, 2 nm to 350 nm, 2 nm to 300 nm, 2 nm to 250 nm, 2 nm to 200 nm, 2 nm to 150 nm, 2 nm to 100 nm, 2 nm to 50 nm, 10 nm to 600 nm, 15 nm to 600 nm, 20 nm to 600 nm, 40 nm to 600 nm, 50 nm to 600 nm, 100 nm to 600 nm, 5 nm to 500 nm, 10 nm to 500 nm, 15 nm to 500 nm, 20 nm to 500 nm, 20 nm to 600 nm, 20 nm to 500 nm, 20 nm to 400 nm, 20 nm to 300 nm, 20 nm to 250 nm, 20 nm to 200 nm, 20 nm to 150 nm, 20 nm to 100 nm, 20 nm to 50 nm, or 20 nm to 40 nm, including any range therebetween. In some embodiments, the size of at least 90% of the nanoparticles varies within a range of less than ±25%, ±20%, ±15%, ±19%, ±5%, including any value therebetween.

Herein throughout, the terms “nanoparticle”, “nano”, “nanosized”, and any grammatical derivative thereof, which are used herein interchangeably, describe a particle featuring a size of at least one dimension thereof (e.g., diameter, length) that ranges from about 1 nanometer to 100 nanometers. Herein throughout, “NP(s)” designates nanoparticle(s).

As used herein the terms “average” or “median” size refer to diameter of the particles. The term “diameter” is art-recognized and is used herein to refer to either of the physical diameter (also termed “dry diameter”) or the hydrodynamic diameter. As used herein, the “hydrodynamic diameter” refers to a size determination for the composition in solution (e.g., aqueous solution) using any technique known in the art, e.g., dynamic light scattering (DLS).

In some embodiments, the dry diameter of the particles, as prepared according to some embodiments of the invention, may be evaluated using transmission electron microscopy (TEM) or scanning electron microscopy (SEM) imaging.

The particle(s) can be generally shaped as a sphere, incomplete-sphere, particularly the size attached to the substrate, a rod, a cylinder, a ribbon, a sponge, and any other shape, or can be in a form of a cluster of any of these shapes, or a mixture of one or more shapes. In some embodiments, the particle has a spherical shape, a quasi-spherical shape, a quasi-elliptical sphere, an irregular shape, or any combination thereof.

In some embodiments, the particle of the invention comprises an oil. In some embodiments, the oil comprises mineral oil, hydrocarbon, fatty acid, mono-, di-, triacylglycerols, vegetable oil, wax, essential oil, aromatic oil, or any combination thereof.

As used herein, the term “oil” refers to any suitable water-immiscible compound. In some embodiments, the oil is an oil that is liquid at room temperature (20° C.; 1013 mbar). In some embodiments, the oil is selected from the group consisting of essential oils, vegetable oils, mineral oils, organic oils, lipids, and any water-immiscible liquids. In some embodiments, the oil is silicone oil.

In some embodiments, the major phase is a water phase. In some embodiments, the oil: water ration is 20:80-40:60.

In some embodiments, the ratio of the major phase and the minor phase is 5:1 to 1:1 (w/w), 4:1 to 1:1 (w/w), 3:1 to 1:1 (w/w), or 2:1 to 1:1 (w/w), including any range therebetween. In some embodiments, the ratio of the major phase and the minor phase is 1:1 (w/w).

Methods

According to some embodiments, there is provided a method for treating or preventing a viral infection in a subject in need thereof, comprising administering to said subject a therapeutically effective amount of the composition of the invention, thereby treating or preventing a viral infection in the subject.

According to some embodiments, the composition of the invention is an immunogenic composition. The term “immunogenic composition” as used herein refers to a composition that is able to produce an immune response.

In some embodiments, the immunogenic composition exhibits, upon administration, activation of T cells. In some embodiments, the immunogenic composition exhibits, upon administration, activation of CD4+ T cells. In some embodiments, the immunogenic composition exhibits, upon administration, activation of CD8+ T cells. In some embodiments, the immunogenic composition exhibits, upon administration, combined activation of CD4+ and CD8+ T cells.

In some embodiments, the immunogenic composition exhibits, upon administration, production of specific antibodies (of any immunoglobin class) against epitopes within the said peptides. Each possibility represents a separate embodiment.

As used herein, the terms “subject,” refers to any subject, particularly a mammalian subject, for whom therapy is desired, for example, a human.

In some embodiments, a subject in need thereof is afflicted with a pathogenic infection. In some embodiments, a subject in need thereof is susceptible to a pathogenic infection. In some embodiments, a subject in need thereof is potentially susceptible to a pathogenic infection.

In some embodiments, the immunogenic composition may be administered to subjects by a variety of administration modes, including by intradermal, intramuscular, subcutaneous, intravenous, intra-atrial, intra-articular, intraperitoneal, parenteral, oral, rectal, intranasal, intrapulmonary, and transdermal delivery, or topically to the eyes, ears, skin or mucous membranes.

For prophylactic and treatment purposes, the composition may be administered to the subject in a single bolus delivery, via continuous delivery (e.g., continuous intravenous or transdermal delivery) over an extended time period, or in a repeated administration protocol (e.g., on an hourly, daily or weekly basis). The various dosages and delivery protocols contemplated for administration of the composition are immunogenically effective to prevent, inhibit the occurrence or alleviate one or more symptoms of infection in the subject. An “immunogenically effective amount” of the peptide thus refers to an amount that is effective, at dosages and for periods of time necessary, to elicit a specific T lymphocyte mediated immune response and/or a humoral response. This response can be determined by conventional assays for T-cell activation, including but not limited to assays to detect antibody production, proliferation, specific cytokine activation and/or cytolytic activity, e.g., using an antibody concentration/titer assay (e.g. via ELISA).

For prophylactic and therapeutic use, peptide antigens might be formulated with a “pharmaceutical acceptable carrier”. As used herein, “pharmaceutically acceptable carrier” includes any and all solvents, dispersion media, coatings, antibacterial and antifungal agents, isotonic and absorption enhancing or delaying agents, and other excipients or additives that are physiologically compatible. In specific embodiments, the carrier is suitable for intranasal, intravenous, intramuscular, intradermal, subcutaneous, parenteral, oral, transmucosal or transdermal administration. Depending on the route of administration, the active compound may be coated in a material to protect the compound from the action of acids and other natural conditions which may inactivate the compound.

According to some embodiments, the present invention provides a method for preparing the composition described herein, comprising the steps of: a. mixing 5-30% (w/w) of the nanoparticles to the major phase, thereby forming a mixture; and b. adding the minor phase to the mixture, and mixing for a period of time.

In some embodiments, mixing is high shear mixing, ultrasonication, overhead stirring, homogenizing, or a combination thereof. In some embodiments, a period of time is 1 min to 24 hour, 5 min to 24 hour, 10 min to 24 hour, 30 min to 24 hour, 1 hour to 24 hour, 2 hour to 24 hour, 3 hour to 24 hour, 5 hour to 24 hour, 6 hour to 24 hour, 1 hour to 12 hour, 2 hour to 12 hour, 3 hour to 12 hour, 5 hour to 12 hour, 6 hour to 12 hour, 1 hour to 8 hour, 2 hour to 8 hour, 3 hour to 8 hour, or 5 hour to 8 hour, including any range therebetween.

In some embodiments, the minor phase comprises 0.5% to 40% (w/w), 0.5% to 30% (w/w), 0.9% to 30% (w/w), 1% to 30% (w/w), 5% to 30% (w/w), 10% to 30% (w/w), 25% to 30% (w/w), 0.5% to 10% (w/w), 0.9% to 10% (w/w), 1% to 10% (w/w), 5% to 10% (w/w), 0.5% to 5% (w/w), 0.9% to 5% (w/w), or 1% to 5% (w/w), of the polymer, including any range therebetween.

In some embodiments, the minor phase comprises 0.5% to 20% (w/w), 0.5% to 15% (w/w), 0.9% to 15% (w/w), 1% to 15% (w/w), 10% to 15% (w/w), 15% to 20% (w/w), 5% to 10% (w/w),), 0.5% to 10% (w/w), 0.9% to 10% (w/w), 1% to 10% (w/w), 5% to 10% (w/w), 0.5% to 5% (w/w), 0.9% to 5% (w/w), or 1% to 5% (w/w), of the active agent, including any range therebetween.

In some embodiments, the ratio of the major phase and the minor phase is 5:1 to 1:1 (w/w), 4:1 to 1:1 (w/w), 3:1 to 1:1 (w/w), or 2:1 to 1:1 (w/w), including any range therebetween. In some embodiments, the ratio of the major phase and the minor phase is 1:1 (w/w).

### General

As used herein the term “about” refers to ±10%.

The terms “comprises”, “comprising”, “includes”, “including”, “having” and their conjugates mean “including but not limited to”.

The term “consisting of means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Chemicals and buffers. All Fmoc protected amino acids, wang resin and hexafluorophosphate azabenzotriazole tetramethyl uronium (HATU) were purchased from Matrix Innovation (Quebec, Canada). N, N′-dimethylformamide (DMF), dichloromethane (DCM), N, N′-diisopropylethylamine (DIPEA), piperidine, methanol, trifluoroacetic acid (TFA), diethyl ether and ethanol were purchased from Bio-Lab (Jerusalem, Israel). Tri isopropyl silane (TIPS), thioanisole, 1,2-ethanedithiol (EDT), acetic anhydride, hydroxybenzotriazole (HOBT), N, N′-diisopropylcarbodiimide (DIC), 5(6)-Carboxyfluorescein [5(6)-FAM] and phenol were purchased from Sigma Aldrich (St. Louis, Mo., USA). Paraffin oil (puriss, meets analytical specification of Ph. Eur., BP, viscous liquid), Dimethylaminopropyl)-N′-ethylcarbodiimide hydrochloride (EDC), MES hydrate were purchased from Sigma-Aldrich. HPLC grade water were purchased from Alfa Aesar and was used as received without further purification. Ultra-sonication (Sonics Vibra-cell ultrasonic liquid processor, Model-VCX 750, Newtown, Conn., USA).

Synthesis of the epitopes according to the sequence of the SARS-CoV-2. The sequence CASYQTQTNSPRRAR (SEQ ID NO: 2) that is unique to the spike glycoprotein of SARS-CoV-2 was used in this study as a model epitope for the SARS-CoV-2. The epitope was synthesized by solid state peptide synthesis (SSPS). See supporting information for peptide synthesis and purification protocol.

Preparation of ToBRFV and PepMV native VLPs. ToBRFV and PepMV co-infected tomato plants (leaves, fruits) were used as a starting material for virus purification as described before (Klap C, et al. Plants 2020, 9(5): 623, and Klap C, et al. Viruses 2020, 12(8): 879). The obtained viral preparation was first visualized by transmission electron microscopy (TEM) to confirm the presence of the characteristic morphology of the two viral particles (tobamovirus and potexvirus). The viral preparation (10 ml) was then mixed with equal volume of 0.01M phosphate buffer pH 9.5 pH (v/v) and the purified viruses were disassembled by incubation at 96° C. for 20 min. RNase A (20 ul, 10 units per μl) was added and the sample was incubated at 96° C. for additional 5 min followed by mild rotations at room temperature for 10 min to allow natural assembly of VLPs.

Functionalization and characterization of the VLPs with the synthetic epitopes. Stock solutions of 57.51 mg of EDC were prepared separately in 10 mL of 0.1 M MES (pH 4.5-5) buffer. The carboxyl groups present in the peptide molecules reacted with the amine groups of the VLP in the presence of EDC to form an amide bond. 112.5 mg of the peptide molecules were added to a 60 mL mixture of 10 ml of the EDC, 50 mL of the VLP (12-15 mg/mL) dispersion. The solution was then mixed by shaker for 2 h at ambient temperature. Subsequently, the mixture was centrifuged and rinsed with MES buffer to remove excess reactants. EDC was used as a cross-linker to covalently immobilize the peptide molecule to the VLP by primarily reacting with the carboxyl groups and producing an amine-reactive O-acylisourea. This intermediate product reacted with the amino groups of the VLP to yield an amide bond, to form the VLP/peptide-epitope conjugates and urea as a by-product38. The VLP/peptide-epitope conjugates were then dispersed again in the water (pH ˜8.5) for further analysis. The same protocol was utilized for the synthesis of VLP/fluorescent peptide conjugates.

Preparation and characterization of o/w Pickering emulsions stabilized by VLPs/epitopes conjugates. Oil-in-water emulsions stabilized by VLPs were prepared by addition of a known amount of paraffin oil (used as received) to VLPs aqueous dispersion (1.3 wt %) at o/w ratios of 20:80, 30:70, 40:60, and 50:50 respectively. Prior to emulsification, the VLPs were dispersed in distilled water (pH ˜8.5) via agitation in a vortex for 2 min. The emulsification was performed by ultrasonication in an ultrasonic probe for 10 minutes at an amplitude of 25%. The emulsions which were stabilized by VLPs/peptide-epitope conjugates and by VLP/fluorescent peptide were prepared by the same aforementioned procedure under the same compositions.

Confocal laser scanning microscopy. Confocal images were collected on a Leica SP8 confocal microscope (Leica Microsystems CMS GmbH, Wetzlar/Germany) equipped with an inverted microscope fitted with a 40×HC PL APO CS2 (1.10 NA) water immersion objective. Excitation of 6-AF and Nile Red was from the 488 nm and the 552 nm laser line of an OPS laser, respectively. The 1024×1024 images were collected using Leica Application Suite X software (Leica Microsystems CMS GmbH, Wetzlar/Germany).

Cryogenic-field emission scanning electron microscopy. Cryogenic-field emission scanning electron microscopy (cryo-FESEM) analysis was performed on a JSM-7800F Schottky Field Emission Scanning Electron Microscope (Jeol Ltd., Tokyo/Japan). Liquid nitrogen was used in all heat exchange units of the cryogenic system (Quorum PP3010, Quorum Technologies Ltd., Laughton/United Kingdom). A small droplet of the freshly mixed emulsions was placed on the sample holder between two rivets, quickly frozen in liquid nitrogen for a few seconds and transferred to the preparation chamber where it was fractured (at −140° C.). The revealed fractured surface was sublimed at −90° C. for 10 min to eliminate any presence of condensed ice and then coated with platinum. The temperature of the sample was kept constant at −140° C. Images were acquired with either a secondary electrons (SE), low electron detector (LED) or backscattered electron (BSE) detector at an accelerating voltage of 1 to 15 kV and a working distance of max. 10.1 mm.

Immunofluorescence detection of VLPs by double labelling. The presence of each virus coat protein in the oil-water interface was confirmed by immunofluorescence using specific primary ToBRFV and PepMV antibodies followed by fluorescent secondary antibodies.

VLPs samples (10-20 μl) were pipetted on poly-lysine coated silicon chips, which were placed in 96 well plates, and incubated for 1 h at room temperature (RT). The un-bound solution was removed and fixation was carried out for 1 h at RT using fixation buffer containing 4% (v/v) formaldehyde, 0.2% (v/v) glutaraldehyde in phosphate-buffered saline (PBS) pH 7.0. Fixation buffer was removed and samples were washed with PBS, 3 times for 10 min each, while rotating at 100 rpm at RT. Blocking was performed with 100 μl PBS containing 2% (w/v) skim milk powder for 30 min at RT. Blocker was removed and samples were incubated with 100 μl specific antisera against ToBRFV (1:4000 dilution in the PBS-milk solution) for overnight at 4° C. while shaking. The samples were washed 3-4 times with PBS pH 7.0 at RT for 10 min each, and 100 μl of the secondary antibody, goat anti-rabbit IgG [conjugated to Alexa Fluor 594 (Invitrogen, Carlsbad, Calif., USA)], were added at a 1:1,000 dilution in PBS and incubated for 3 h at 37° C. with agitation at 100 rpm. The samples were then washed 3-4 times with PBS pH7.0 for 10 min each. In order to block all unbound ToBRFV antibodies 100 μl of a high concentration of unlabelled AP conjugated goat anti-rabbit antibodies (SIGMA, A9919, 1:100 dilution in PBS containing 2% non-fat milk) were added and samples were incubated for 3 h at 37° C. Washes (×3-4) with PBS pH 7.0 were carried out at RT for 10 min each with agitations. Blocking solution (100 μl PBS containing 2% non-fat milk) was added and samples were incubated for 30 min at RT with agitation. Blocker was removed and 100 μl specific antisera against PepMV (1:8,000 dilution in the PBS-milk solution) were added for overnight at 4° C. while shaking. Washes (×3-4) with PBS pH 7.0 were carried out at RT for 10 min each with agitation and 100 μl goat anti-rabbit IgG [conjugated to Alexa Fluor 488 (Invitrogen, Carlsbad, Calif., USA)], were added at a 1:1,000 dilution in PBS and incubated for 3 h at 37° C. with agitation at 100 rpm. Washes (×3-4) with PBS pH 7.0 were carried out at RT for 10 min each with agitation and samples were kept in 1004, PBS pH 7.0 in sealed plates at 4° C.

In-vivo preclinical trial in mice. To evaluate the immunogenicity of the tested items, we employed a standard vaccination scheme in Balb/C mice as outlined in the illustration. Groups of 7, 6-7 weeks old female mice were immunized via SC route with test items or controls, at day 1, and boosted on days 14 and 28 with blood drawn before immunization, at termination. Samples were processed, sera collected and analysed for anti-epitope reaction in a standard direct ELISA assay. This study was performed in compliance with “The Israel Animal Welfare Act” and following “The Israel Board for Animal Experiments”. The following eight groups were immunized: (Epitope 1 only); (Epitope 1 in emulsion); (Epitope 2 only); (Epitope 2 in emulsion); (Epitope 3 only); (Epitope 3 in emulsion); (Emulsion only); (Carrier VLP only).

Evaluation parameters. Morbidity & Mortality-Twice daily (once daily over the weekend). Body Weight Monitoring-During acclimation, weekly thereafter. Blood Draws-baseline, and at termination (all mice). Blood Processing-Blood from all mice was collected at termination. Blood samples were processed into serum/for detection of antibodies to the antigen by ELISA. Method Development & antibody titer evaluation-Generation of antibodies (IgG) against the antigen was detected by direct ELISA in sera of immunized mice.

IgG quantification by direct ELISA. Generation of antibodies was detected by ELISA. The purpose of this ELISA was to ascertain that the mice elicited an immune response against the antigen. On Day 1 Three 96 well ELISA plates were coated with 25 μL of VLP-peptide at 2.5 mg/mL (250 μg/100 μL) in Carbonate/Bicarbonate Buffer (Sigma, Cat #C3041). The plates were incubated for 2.5 hours at 37° C. The coating solution was removed and the plates were washed three times with wash solution (PBS/0.05% tween), with 1-minute incubation between washes. 50 μL of blocking buffer (1% BSA in PBS) were added and the plates were incubated overnight at 4° C. On Day 2 The blocking buffer was removed and the plates were washed three times with wash solution (PBS/0.05% tween), with 1-minute incubation between washes. 25 μL of 1:1000, 1:10000, and 1:50000 serum samples (diluted in PBS/0.1% BSA) and blank (PBS/0.1% BSA only) were added, in duplicates and the plates were incubated over night at 4° C. On Day 3 The samples were removed and the plates were washed three times with wash solution (PBS/0.05% tween), with 1-minute incubation between washes. 25 μL of secondary antibody (Peroxidase AffiniPure Donkey Anti-Mouse IgG (H+L) Cat 715-035-151) were added and the plates were incubated for 2 hours at 37° C. The samples were removed, and the plates were washed three times with wash solution (PBS/0.05% tween), with 1-minute incubation between washes. 25 μL of TMB substrate were added to each well and the plates were incubated for 15 min at room temperature or until the desired colour was achieved. 25 μL of Stop Solution were added to each well before reading the plates. The plates were read at 450 nm using a microplate reader.

Peptide synthesis: The peptide, Ac-NH-Arg-Ala-Arg-Arg-Pro-Ser-Asn-Thr-Gln-Thr-Gln-Tyr-Ser-Ala-Cys-OH and its 5(6)-FAM-labeled (Ex: 492 nm, Em: 514 nm) peptide were synthesized using wang resin having a substitution level of 0.83 mmol/g. 300 mg of wang resin was swelled in a mixture of DMF and DCM (1:1) overnight prior to the synthesis. Each coupling reaction was performed using 5 equivalents of HATU as activator, 5 equivalents of amino acids and 10 equivalents of DIPEA as the activator base. The concentration of amino acids and HATU in the coupling mixture was 0.2M. The arginine amino acid which is after proline was coupled two times. DMF was used as solvent. The Fmoc deprotection was performed by 20% piperidine solution in DMF.

Acetyl protection at the N-terminal: The resin with free N-terminal of the peptide was treated with the mixture of acetic anhydride, HOBT and DIPEA in DMF and stirred for 3 hours. It was performed twice to ensure complete N-terminal acetyl protection.

5(6)-FAM protection at the N-terminal: The resin with free N-terminal of the peptide was treated with the mixture of 5(6)-FAM, HOBT and DIC in DMF and stirred for 48 hours. It was performed twice to ensure complete N-terminal 5(6)-FAM protection.

After synthesizing the whole peptide, the resin was washed by DMF (5 times), DCM (5 times), methanol (5 times), diethyl ether (5 times) and was kept under a high vacuum pump for 4 h for complete drying. The resin containing peptide was treated with cleavage cocktail containing TFA (92%), TIPS (1.5%), water (2%), thioanisole (1.5%), 1,2-ethanedithiol (1.5%) and phenol (1.5%) for 24 hours at room temperature under shaking. The cleavage solution (without resin) was collected into a 50 mL falcon tube and was evaporated to minimum volume using flow of N₂. The residue solution was poured into ice-cold diethyl ether for precipitation. It was then stored overnight at −20° C. Next, it was centrifuged at 5000 rpm at 4° C. and the precipitate was dissolved in triple distilled water (TDW). The peptide was lyophilized to obtain a white solid powder.

Peptide purification and characterization: The peptide was purified by reverse phase preparative high-performance liquid chromatography (HPLC) using Thermo Scientific Ultimate 3000 system with a C18 LC column (10 μm, 110 Å, 250×21.2 mm). A linear gradient (5% to 95%) flow of acetonitrile (with 0.1% TFA) with time in water (with 0.1% TFA) at a flow rate of 10 ml/min was used to elute peptide and each fraction were characterized by electron spray ionization mass spectroscopy using an LCQ Fleet Ion Trap mass spectrometer (Thermo Fisher Scientific, Waltham, Mass. USA). The purity was checked by analytical reversed phase high-performance liquid chromatography (HPLC) using Waters e2695 separation module with a C18 LC column (5 μm, 110 Å, 250×4.6 mm). A linear gradient (5% to 95%) flow of acetonitrile (with 0.1% TFA) with time in water (with 0.1% TFA) at a flow rate of 1 ml/min was used to elute peptide. UV detection at 220 nm was used to monitor the peptide flow through column.

Western blot analysis: Samples of the symptomatic tomato fruits (pericarp, seeds, juice mesocarp, exocarp) and mechanically inoculated tomato plants (leaves) were weighed and subjected to protein extraction by suspending the weighed samples in USB buffer containing 75 mM Tris-HCL (pH6.8), 8 M urea, 4.5% (g/v) SDS and 7.5% (v/v) B-mercaptoethanol while keeping constant μg/μl ratios in all samples. The extraction was carried out by crushing the fruit samples in the USB buffer, or mixing the fruit juice with the USB buffer and incubating the suspensions at 90° C. for 15 min. The suspensions were centrifuged at 14,000 g for 15 min and the supernatants were subjected to western blot analysis. Samples were separated on 15% SDS-PAGE. The gels were electro-blotted onto a nitrocellulose membrane for 30 min at 200 mAmp (for a single gel) using a semi-dry transfer apparatus (Bio-Rad). The membrane was blocked for 2 h at room temperature with 3% non-fat dry milk in PBS and the specific antisera for ToBRFV or PepMV was added for overnight incubation at 4° C. The alkaline phosphatase (AP) conjugated goat anti-rabbit antibodies (Sigma) were used for detection with the addition of AP-substrate NBT, BCIP (Bio-Rad).

Virus purification: Purification of viral particles was performed using 100 g symptomatic tomato fruits and leaves crushed in 100 ml 0.1 M potassium phosphate buffer, pH=7.0 containing 0.5% sodium sulphite. Chloroform-Butanol mixture (1:1, v/v) comprising 10% of the fruit solution volume was added and the total mixture was incubated 1 h at 4° C. After centrifugation at 13,000 g for 20 min the supernatant was filtered through Miracloth (Cal-Biochem) and the filtrate was ultra-centrifuged at 200,000 g for 2.5 h. The pellet was suspended in 1 ml 0.01 M potassium phosphate buffer pH=7.0 and placed on 4 ml sucrose 30% in 0.01 M potassium phosphate buffer pH=7.0. Clean virus preparation was pelleted by ultra-centrifugation at 200,000 g for 2.5 h. TEM analysis was performed using 2% uranyl acetate and visualization in an FEI Tecani T12, equipped with Gatan ES500W Erlangshen camera.

Particle disassembly to generate Virus Like Particles (VLPs): 400 μl of virus preparation from ToBRFV and PepMV infected tomatoes' fruit and leaves (1.3 mg/ml) served for this small-scale protocol. The virus preparation sample was incubated at 96° C. for 20 min, immediately RNAse A or H (100μ/μl) was added to the sample in order to degrade the two viral RNAs and to prevent the natural assembly of virus particles allowing the generation of the VLPs structures.

### Example 1

### Self-Assembly of VLPs

The purification of the viral particles was performed using 100 gr symptomatic tomato fruits and leaves, as described by Luria et al. (Luria N, et al. A New Israeli Tobamovirus Isolate Infects Tomato Plants Harboring Tm-22 Resistance Genes. PloS one 2017, 12(1): e0170429-e0170429).

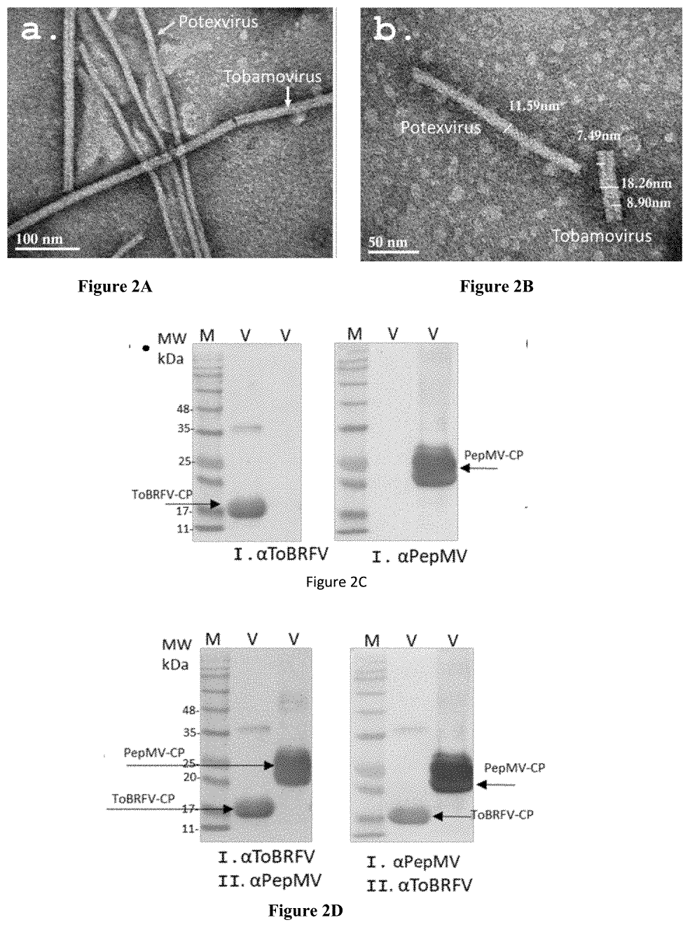

FIGS. 2A, B depicts transmission electron microscopy (TEM) characterization of virus preparations from ToBRFV and PepMV infected symptomatic tomato plants. The rod-like and filamentous particle structures of ToBRFV and PepMV, respectively can be visualized. Western blot analyses showed the presence of ToBRFV and PepMV in the viral preparation from the co-infected tomato plants (FIG. 2C, D). ToBRFV-CP of ˜17.5 kDa and PepMV-CP of ˜26 kDa were specifically detected and the presence of both viruses in each tested viral preparation was confirmed (FIG. 2C, D).

The viral preparation samples were incubated at 96° C. for 20 min for viral disassembly, and immediately RNAse A or H were added to the samples for degradation of the two viral RNAs and preventing the natural reassembly of the native viral particles allowing the generation of the new VLP structures.

### Example 2

### Synthesis of the VLP/Epitope Conjugates

The C-terminus of the spike glycoprotein (SG) of SARS-CoV-2 contains an additional unique amino acid sequence that is absent in other coronaviruses. It was suggested to be involved in the pathogenicity of the virus and could be therefore targeted for the development of antiviral immunity (Coutard B, et al. Antiviral Research 2020, 176: 104742). This amino acid sequence: CASYQTQTNSPRRAR (SEQ ID NO: 2), was used in this study as a model epitope for vaccine preparation against SARS-CoV-2.

The epitope was synthesized by simple solid-state peptide synthesis (SSPS). The resulting synthetic peptide has an acetyl group at the N-termini and amine at the C-termini, enabling to immobilize it on the VLPs at the required directionality in accordance with the spike glycoprotein. Using simple cross-linking chemistry by 1-ethyl-3-(3-dimethylaminopropyl)-carbodiimide hydrochloride (EDC), the peptide was covalently immobilized on the VLPs by amidation through their amine which reacts with available carboxylic groups on the VLPs. The resulting VLP/epitope conjugates were purified by ultra-centrifugation and served as stabilizers at the o/w interphase of oil droplets in oil-in-water Pickering emulsions for further enhancement of epitope presentation (see, Example 3 below).

### Example 3

### Plant Virus VLPs could Serve as Stabilizers of Pickering Emulsions

ToBRFV and PepMV derived VLPs were tested as effective stabilizers of oil-in-water Pickering emulsions using paraffin as the oil phase due to its well-established biocompatibility in many other vaccine formulations. Emulsions stabilized by VLPs were prepared by addition of paraffin oil to VLPs aqueous dispersion (1.3 wt %) at o/w ratios of 20:80, 30:70, 40:60, and 50:50 respectively. Prior to emulsification, the VLPs were dispersed in water via agitation in a vortex for 2 min. The emulsification was performed by ultrasonication in an ultrasonic probe for 10 minutes at an amplitude of 25%. Uniform emulsions were obtained at any of the aforementioned compositions.

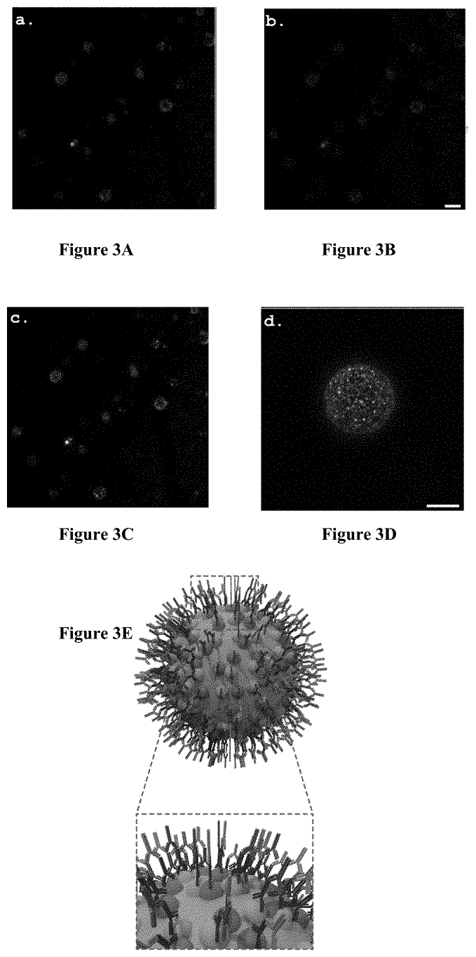

Visualization of the VLPs by immunofluorescence was carried out by subjecting the emulsions to ToBRFV and PepMV CP detections using specific primary antibodies followed by the secondary fluorescent antibodies using Alexa Fluor 488 for ToBRFV (green, FIG. 3A) and Alexa Fluor 594 for PepMV (red, FIG. 3B). The fluorescent signals of both ToBRFV and PepMV CPs were located at the interface of the oil droplets confirming assembly of both VLPs at the o/w interface stabilizing the Pickering emulsion. Visualization of the fluorescent signals using both red and green channels showed that the two different VLP types homogenously shared the interface (FIG. 3C, D).

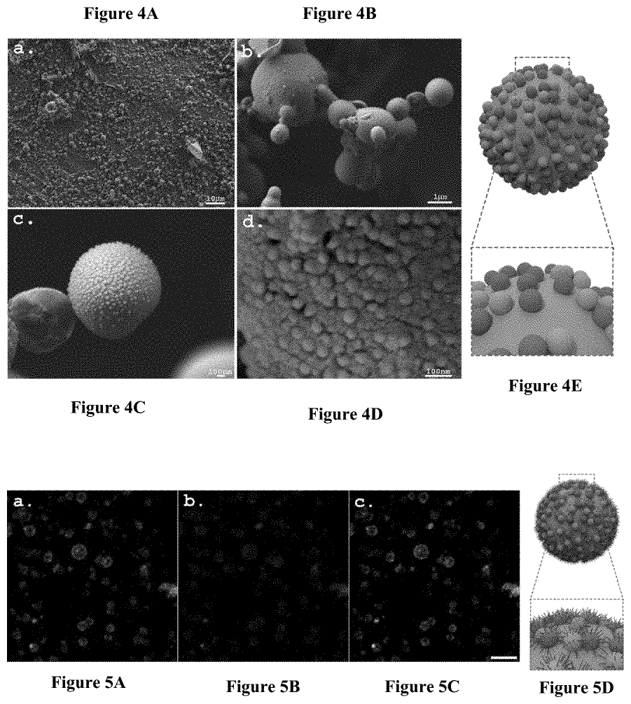

The structure of the emulsions, and in particular the nanostructure of the interface, was studied by cryo-HRSEM. The Pickering emulsions were vitrified using liquid nitrogen and then fractured. The vitrification procedure enabled us to directly observe the nanostructure of the interface since no structural changes took place during vitrification. In the second stage, the continuous phase of the emulsions (i.e., the water) was sublimated, revealing the interface, which made it possible to study its nanostructure. FIGS. 4A-D depict characteristic cryo-HRSEM micrographs of a 20:80 o/w emulsion stabilized by VLPs at a concentration of 1.3% wt. A basic structure of a Pickering emulsion was observed, confirming the formation of a paraffin o/w emulsion (FIG. 4A). At higher magnifications, a layer of nanoparticles decorating the surface of the oil droplets at the o/w interface was observed. The particle diameter range was 20-50 nm, corresponding to the expected diameter of the VLPs (FIG. 4C, D). The cryo-HRSEM direct observation results conclusively confirmed the successful assembly of the VLPs at the interface of the oil droplets. Moreover, it could be seen that both types of VLPs had similar spherical structures.

### Example 4

### Development of Pickering Emulsions Stabilized by VLP/Epitope Conjugates

ToBRFV and PepMV derived VLPs, successfully stabilizing paraffin oil-in-water Pickering emulsions, could indicate that the newly designed platform would allow enhanced presentation of SARS-CoV-2 S1 epitopes by using VLP/epitope conjugates as Pickering stabilizers. A fluorescent [5(6)-FAM] labelled SARS-CoV-2 S1 unique peptide that was covalently immobilized on the VLPs was designed. The VLP/[5(6)-FAM] peptide conjugates, dispersed in water at 1.3 wt %, were engaged as stabilizers of paraffin oil-in-water Pickering emulsions prepared by using four different o/w ratios of 20:80, 30:70, 40:60, and 50:50. The emulsification procedure and the compositions were identical to the one used for the VLPs based emulsions.

The resulting [5(6)-FAM] labelled conjugate-based Pickering emulsions were uniform at any of the studied o/w ratios. Visualizations of fluorescent [5(6)-FAM] labelled epitope/VLP conjugates in the Pickering emulsions with the green channel by confocal fluorescence microscopy clearly showed the green fluorescence of [5(6)-FAM] labelled epitope located at the o/w interface of the oil droplets (FIG. 5A). The specific fluorescent signal of PepMV-CP that comprised the VLPs, visualized with the red channel (FIG. 5B), and co-localization of the red and green fluorescent signals, visualized with both red and green channels (FIG. 5C, shown as orange signals), have confirmed that the peptide epitopes covalently immobilized on the VLPs, which were assembled on the oil droplet surface, present the predicted enhanced pathogenic epitope presentation when using the designed vaccine development formulation.

### Example 5

### In Vivo Immunogenicity Assay of the Studied VLP/Epitope-Based Emulsions

To evaluate the immunogenicity of the studied VLP/epitope-based emulsions, the inventors have employed a standard Balb/C mice vaccination scheme. Blood samples were collected from the mice and the sera were tested for detection of IgG antibodies against the peptide antigen using ELISA. Three different dilutions of the serum were studied: 1:1,000, 1:10,000 and 1:50,000.

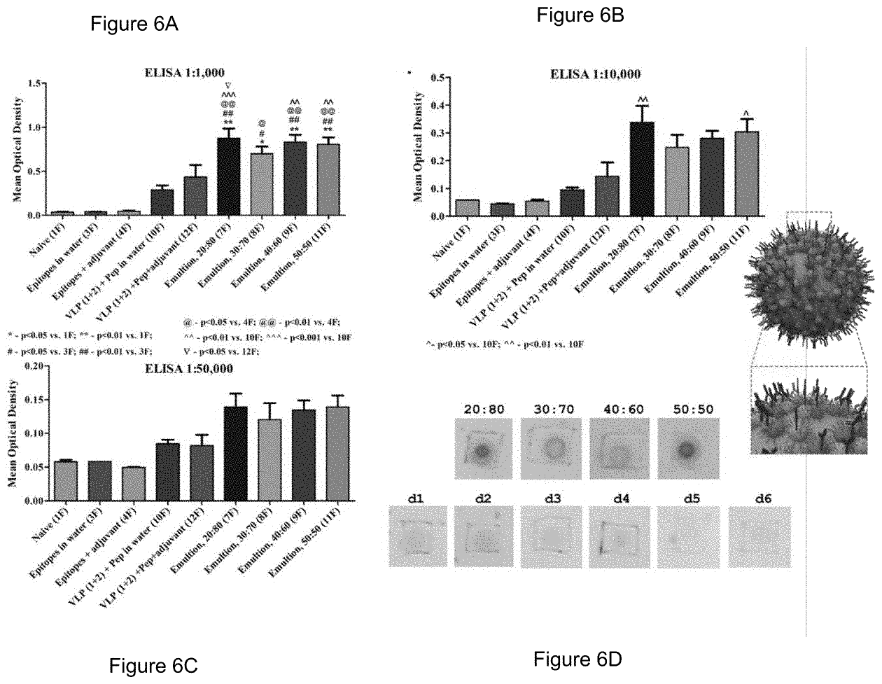

The αSARS-CoV-2-S1 IgG titers of the studied mouse antisera developed against the SARS-CoV-2-S1-peptide under different epitope preparation conditions showed an order of magnitude higher IgG titers in the studied VLP based emulsions compared to epitopes dissolved in water and epitopes administered with an adjuvant (FIG. 6A, B). In addition, the assembly of VLP/epitope conjugates at the oil/water interface, stabilizing the Pickering emulsions, showed two times higher IgG titers compared to the non-assembled VLP/epitope conjugates (aqueous dispersions of VLP/epitope conjugates) (FIG. 6A, B). These results conclusively confirm the ability to obtain two-mode enhancement of the SARS-CoV-2 S1 epitope presentation for the development of a new subunit vaccine formulation against SARS-CoV-2 (FIG. 6A, B). Specificity of the mouse antisera produced in the vaccinated mice against SARS-CoV-2-S1 peptide was confirmed using dot-blot analyses of the various antisera obtained by vaccinations by VLP/epitope stabilized Pickering emulsions prepared by using four different o/w ratios of 20:80, 30:70, 40:60, and 50:50. The results showed a clear indication of a higher production rate of the αSARS-CoV-2-S1 peptide in the studied emulsions (FIG. 6D) compared to epitopes dissolved in water or epitopes administered with an adjuvant (FIG. 6D, d3 and d4, respectively). These results conclusively confirmed that the two-mode enhancement mechanism presentation of the invention, including but not limited to SARS-CoV-2 S1 epitope, could serve as an efficient vaccine. Applying this model for specific immunization against SARS-CoV-2 requires a combination of several epitopes in our described vaccine development platform.

In this study, the inventors have presented a novel technology for the generation of fully synthetic subunit vaccines. The inventors have shown a successful immunization against SARS-CoV-2 S1 in mice. The high intensity of the epitope presentation levels was achieved by a two-mode enhancement mechanism, achieving heterogeneity and density of epitope presentation. The first mode engaged a high epitope concentration of SARS-CoV-2 S1 peptide epitope covalently immobilized on the VLPs surface. The second mode is achieved by the assembly of the VLPs/epitope conjugates on the surface of oil droplets at an oil/water interface of an emulsion as Pickering stabilizers. This two-mode epitope display system provoked a higher IgG titer in mice compared to the classical adjuvant-associated immunization. ToBRFV and PepMV based VLPs are characterized with very high stability which opens up the possibility to develop safe vaccine technologies with improved efficiency and shelf life against various pathogens, including but not limited to, SARS-CoV-2. The described platform is highly flexible and by using multiple epitopes can be easily applied and extended for immunization against a wide range of pathogen-epitopes.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A particle in a form of a colloidosome comprising a shell and a core comprising an oil, wherein said shell comprises an immunogenic nanoparticle in contact with the core, the immunogenic nanoparticle being covalently bound to at least one epitope.

2. The particle of claim 1, wherein said particle has a diameter of 10 μm to 100 μm.

3. The particle of claim 1, wherein said immunogenic nanoparticle has a diameter of 10 nm to 100 nm.

4. The particle of claim 1, comprising 1% to 20% (w/w) of said nanoparticles.

5. The particle of claim 1, wherein said immunogenic nanoparticle is virus-like particle.

6. The particle of claim 5, wherein said virus-like particle is a plant viruses-like particle.

7. The particle of claim 1, wherein said virus-like particle is derived from a coat protein of a virus selected from the group consisting of Tobamovirus, and Potexvirus, or a combination thereof.

8. The particle of claim 1, comprising at least two types of immunogenic nanoparticles.

9. The particle of claim 1, wherein the at least one epitope is derived from SARS-CoV-2 spike glycoprotein.

10. The particle of claim 1, wherein the at least one epitope comprises the amino acid sequence as set forth in TQTNSPRRAR (SEQ ID NO: 1)

11. The particle of claim 10, wherein the at least one epitope is selected from the group consisting of CASYQTQTNSPRRAR (SEQ ID NO: 2); CASYQTQTNSPRRARSV (SEQ ID NO: 3), and ASYQTQTNSPRRARSVAS (SEQ ID NO: 4).

12. The particle of claim 1, wherein the at least one epitope comprises N-terminal acetylation.

13. A composition comprising a plurality of particles of claim 1 and a pharmaceutically acceptable carrier.

14. The composition of claim 13, wherein said composition is an oil-in-water emulsion.

15. A method for treating or preventing a viral infection in a subject in need thereof, comprising administering to said subject a therapeutically effective amount of the composition of claim 13, thereby treating or preventing an infection in the subject.
